(19) United States
(12) Patent Application Publication
(10) Pub. No.: US 2008/0280878 A1
(43) Pub. Date: Nov. 13, 2008

(54) Methods and Compositions for Treating Chronic Lymphocytic Leukemia

(76) Inventors: Januario E. Castro, San Diego, CA (US); Thomas J. Kipps, Rancho Santa Fe, CA (US); Francis J. Burrows, Solana Beach, CA (US); Adeela Kamal, Encinitas, CA (US); Carlos E. Prada, La Jolla, CA (US)

Correspondence Address: WILSON SONSINI GOODRICH & ROSATI, 650 PAGE MILL ROAD, PALO ALTO, CA, 94304-1050, US

(21) Appl. No.: 11/667,005

(22) PCT Filed: Nov. 2, 2005
(86) PCT No.: PCT/US2005/039816

§ 371 Date: Jul. 2, 2008

Related U.S. Application Data
(60) Provisional application No. 60/624,638, filed on Nov. 2, 2004.

(51) Int. Cl. A61K 31/397; A61K 31/33; A61P 35/02

(52) U.S. Cl. 514/210.21; 514/183

(57) ABSTRACT

Novel method of treating chronic lymphocytic leukemia by the administering of HSP90 inhibitors, particularly ansamycins, more particularly I 7-allylamino-I 7-demethoxygetdanarnycin (17-AAG).

### FIELD OF INVENTION

The invention relates in general to treatment of chronic lymphocytic leukemia (CLL), particularly to the treatment of aggressive CLL using HSP90 inhibitors; more particularly to the treatment of CLL using ansamycins, e.g., 17-allylamino-17-demethoxygeldanamycin (17-AAG).

### BACKGROUND

The following description includes information that may be useful in understanding the present invention. It is not an admission that any of the information provided herein is prior art or relevant to the presently claimed inventions, or that any publication specifically or implicitly referenced is prior art.

The course of chronic lymphocytic leukemia (CLL) is variable. In aggressive disease, CLL cells usually express an unmutated immunoglobulin heavy-chain variable-region gene (IgV_{H}) and a 70-kD zeta-associated protein (ZAP70), whereas in indolent disease, the CLL cells usually express mutated IgV_{H} but lack expression of ZAP70. The expression of ZAP70 in CLL patients correlates with disease progression, poor clinical outcome, decreased overall survival and early requirement for treatment. Although the presence of an unmutated IgV_{H} is strongly associated with the expression of ZAP70, ZAP70 is a stronger predictor of the need for treatment and prognosis in B-cell CLL. Rassenti, L. Z. et al., N Eng J Med., 2004, 351, 893-901.

ZAP70 is a 70-kD cytoplasmic protein tyrosine kinase (PTK) that ordinarily is expressed only in natural killer (NK) cells and T-cells and plays a critical role in T-cell-receptor signaling. Keating et al. Hematology, 2003, 153-175. B-cells lack ZAP70, and instead use another related PTK for signal transduction via the B-cell receptor (BCR) complex. Studies found that CLL B cells that have unmutated IgV_{H} genes generally expressed levels of ZAP70 protein that were comparable to those expressed by normal blood T cells. In contrast, CLL B cells that expressed mutated IgV_{H} genes, or that had low-level expression of CD38, generally do not express detectable levels of the ZAP70 protein. Chen, et al. Blood 2002, 100:13, 4609-4614. B-cell expression of ZAP70 is not genetically predetermined. Chen, 2002, supra. Expression of ZAP70 has functional significance for the signaling capacity of the BCR complex expressed in CLL. Keating et al. supra. ZAP70 promotes phosphorylation of downstream signaling molecules after engagement of the BCR and plays a role in membrane antigen-receptor signaling pathways. Keating et al. supra and Rassenti et al. supra. One study shows that expression of ZAP70 in CLL allows for more effective IgM-signaling in CLL B cells, a feature that could contribute to the relatively aggressive clinical behavior generally associated with CLL cells that express unmutated IgVH. Chen, et al. Blood, 2004, prepublication online Oct. 28, 2004.

The eukaryotic heat shock protein 90s (HSP90s) are ubiquitous chaperone proteins involved in folding, activation and assembly of a wide range of client proteins, including mediators of signal transduction, cell cycle control and transcriptional regulation. In order to exert its function on client proteins, HSP90 requires the formation of an active protein complex composed of cochaperone molecules and an active ATP binding site. Proteins identified as HSP90 client proteins include transmembrane tyrosine kinases [HER-2/neu, epidermal growth factor receptor (EGFR), MET and insulin-like growth factor-1 receptor (IGF-1R)], metastable signaling proteins (Akt, Raf-1 and IKK), mutated signaling proteins (p53, Kit, Flt3 and v-src), chimeric signaling proteins (NPM-ALK, Bcr-Abl), steroid receptors (androgen, estrogen and progesterone receptors), cell-cycle regulators (cdk4, cdk6) and apoptosis related proteins. It has been postulated that malignant progression and cancer prognosis may be associated with the presence of activated HSP90 which exists in heightened complexes with cochaperone proteins. Kamal et al., Nature, 2003, 425:407-410.

Ansamycin antibiotics, e.g., herbimycin A (HA), geldanamycin (GDM), 17-AAG, and other HSP90 inhibitors are thought to exert their anticancerous effects by tight binding of the N-terminus ATP-binding pocket of HSP90 (Stebbins, C. et al., Cell, 1997, 89:239-250). This pocket is highly conserved and has weak homology to the ATP-binding site of DNA gyrase (Stebbins, C. et al., supra; Grenert, J. P. et al., J. Biol. Chem. 1997, 272:23843-50). Further, ATP and ADP have both been shown to bind this pocket with low affinity and to have weak ATPase activity (Proromou, C. et al., Cell, 1997, 90: 65-75; Panaretou, B. et al., EMBO J., 1998, 17: 482936). In vitro and in vivo studies have demonstrated that occupancy of this N-terminal pocket by ansamycins and other HSP90 inhibitors alters HSP90 function and inhibits protein folding. At high concentrations, ansamycins and other HSP90 inhibitors have been shown to prevent binding of protein substrates to HSP90 (Scheibel, T., H. et al., Proc. Natl. Acad. Sci. USA, 1999, 96:1297-302; Schulte, T. W. et al. J. Biol. Chem. 1995, 270:24585-8; Whitesell, L. et al. Proc. Natl. Acad. Sci. USA, 1994, 91:8324-8328). Ansamycins have also been demonstrated to inhibit the ATP-dependent release of chaperone-associated protein substrates (Schneider, C. L. et al., Proc. Natl. Acad. Sci. USA, 1996, 93:14536-41; Sepp-Lorenzino et al. J. Biol. Chem. 1995, 270:16580-16587). In either event, the substrates are degraded by a ubiquitin-dependent process in the proteasome (Schneider, C. L. supra; Sepp-Lorenzino, L. et al. J. Biol. Chem., 1995, 270:16580-16587; Whitesell, L. et al., supra).

This substrate destabilization occurs in both tumor and non-transformed cells alike and has been shown to be especially effective on a subset of signaling regulators, e.g., Raf (Schulte, T. W. et al., Biochem. Biophys. Res. Commun. 1997, 239:655-9; Schulte, T. W. et al. J Biol. Chem. 1995, 270:24585-8), nuclear steroid receptors (Segnitz, B., and U. Gehring, J. Biol. Chem. 1997, 272:18694-18701; Smith, D. F. et al. Mol. Cell. Biol. 1995, 15:6804-12 ), v-src (Whitesell, L., et al., Proc. Natl. Acad. Sci. USA, 1994, 91:8324-8328) and certain transmembrane tyrosine kinases (Sepp-Lorenzino, L. et al. J. Biol. Chem. 1995, 270:16580-16587) such as EGF receptor (EGFR), Her2/Neu (Hartmann, F. et al. Int. J. Cancer 1997, 70:221-9; Miller, P. et al., Cancer Res. 1994, 54:2724-2730; Mimnaugh, E. G. et al. J. Biol. Chem. 1996, 271:22796-801; Schnur, R. et al., J. Med. Chem. 1995, 38:3806-3812), CDK4, and mutant p53 (Erlichman et al., Proc. AACR, 2001, 42, abstract 4474). The ansamycin-induced loss of these proteins leads to the selective disruption of certain regulatory pathways and results in growth arrest at specific phases of the cell cycle (Muise-Heimericks, R. C. et al. J. Biol. Chem., 1998, 273:29864-72), and apoptosis, and/or differentiation of cells so treated (Vasilevskaya, A. et al., Cancer Res., 1999, 59:3935-40).

Because ZAP70 expression is associated with an aggressive form of CLL, a means of controlling such an overexpression is needed. A treatment that could simultaneously avoid or minimize harm to normal cells and tissues would be most desirable. The present invention addresses these needs.

### SUMMARY OF THE INVENTION

The inventors of the present invention have found that ZAP70 is a client protein of HSP90 and that specific inhibitors of HSP90, such as 17-AAG, down modulate the expression and function of this tyrosine kinase and induce apoptosis preferentially in ZAP-90 positive CLL B cells in a dose- and time-dependent manner.

One aspect of the invention is a method of treating a form of CLL which is characterized by the expression of ZAP70 in the CLL B cells by administering to a patient in need thereof a pharmaceutically effective amount of a HSP90 inhibitor.

In one embodiment, the inhibitor is an ansamycin; and the ansamycin is selected from the group below, or a polymorph, solvate, ester, tautomer, enantiomer, pharmaceutically acceptable salt or prodrug thereof:

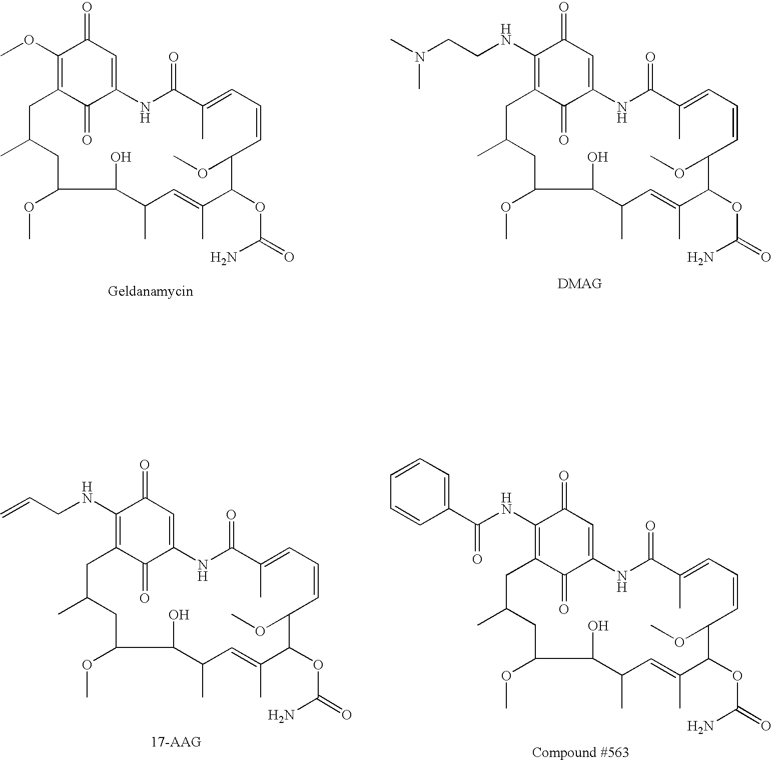

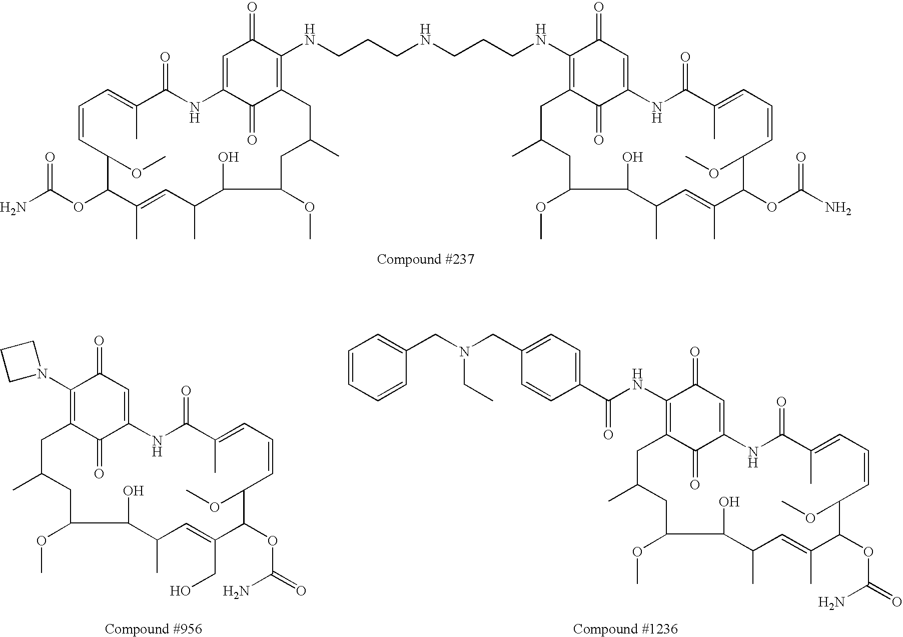

In one further embodiment, the ansamycin is 17-AAG which may comprise low melt forms of 17-AAG characterized by DSC melting temperatures below 175° C. and/or by an X-ray powder diffraction pattern having peaks located at 5.85 degree, 4.35 degree and 7.90 degree two-theta angles. In another embodiment, the ansamycin is a low melt polymorph of 17-AAG which is characterized by a DSC melting temperature at about 156° C. and by an X-ray powder diffraction pattern having peaks located at 5.85 degree, 4.35 degree and 7.90 degree two-theta angles. In yet another embodiment, the ansamycin is another low melt polymorph of 17-AAG characterized by a DSC melting temperature at about 172° C. Further, the 17-AAG may be a high melt form, a low melt form, an amorphous form, or combination thereof.

In yet other embodiment, the inhibitor binds at the ATP-binding site of a HSP90.

In another aspect of the invention, the HSP90 inhibitor is administered intravenously, intralesionally, parenterally, or orally.

In a further aspect of the invention, the HSP90 inhibitor has an IC₅₀ between about two to 10 fold lower for the HSP90 in the B cells of the patient having elevated ZAP70 than the HSP90 in the normal B cells that do not have elevated ZAP70. In one embodiment, the HSP90 inhibitor has an IC₅₀ about two fold, 5 fold or 10 fold lower for the HSP90 in the B cells of the patient having elevated ZAP70 than the HSP90 in the normal B cells that do not have elevated ZAP70.

In another aspect of the invention, the inhibitor exhibits an IC₅₀ of about 100 nM or less in the cells having elevated ZAP70. In one embodiment, the inhibitor exhibits an IC₅₀ of about 75 nM or less in the cells having elevated ZAP70. In one embodiment, the inhibitor exhibits an IC₅₀ of about 50 nM or less in the cells having elevated ZAP70. In a further embodiment, the inhibitor exhibits an IC₅₀ of about 30 nM in the cells having elevated ZAP70.

The above aspects and embodiments may be combined when feasible or appropriate. Other aspects and variation of the forgoing aspects and embodiments which are obvious to those skilled in the art are within the contemplation of the invention.

Advantages of the invention include one or more of ease of manufacture, the use of clinically acceptable reagents (e.g., having reduced environment and/or patient toxicity), enhanced formulation stability, less complicated shipping and warehousing, and simplified pharmacy and bed-side handling. Other advantages, aspects, and embodiments will be apparent from the description above and the detailed description and claims to follow.

### BRIEF DESCRIPTION OF THE DRAWINGS

FIG. 1 shows the competitive binding of 17-AAG against a biotinylated geldanamycin probe (biotin-GM) for HSP90 in lysates of B cells, T cells, ZAP70 positive CLL B cells (ZAP70+ CLL B cells) and ZAP70 negative CLL B cells (ZAP70− CLL B cells). The Western blot bands show that inhibition of binding of HSP90 to the biotin-GM decreases with increasing concentration of 17-AAG (1a.). The results are quantitated and plotted in % inhibition of binding of HSP90 to the biotin-GM vs. 17-AAG concentration in nM (1b). The IC₅₀ reported is the concentration of 17-AAG needed to cause half-maximal inhibition of binding (1c).

FIG. 2 presents graphically the inhibition of binding of biotinylated geldanamycin probe (biotin-GM) for HSP90 in lysates of ZAP70+ CLL B cells (▴) and ZAP70− CLL B cells (▪) in the presence of increasing concentrations of 17-AAG.

FIG. 3 presents graphically the inhibition of binding of biotinylated geldanamycin (biotin-GM) for HSP90 in lysates of normal B cells (♦) and T cells (▪).

FIG. 4 demonstrates the association of HSP90 and ZAP70 in MCF-7 breast carcinoma cells, ZAP70+ CLL B and ZAP70− CLL B and normal T and B cells by co-immunoprecipation and analyzed by SDS-PAGE and Western blots using the indicated antibodies. “IP” denotes immunoprecipation and “WB” denotes Western Blot. P23 and HOP are essential components of two known multi-chaperone HSP90 complexes.

FIG. 5 compares the degradation of ZAP70 in ZAP70+CCL B cell after treatment with EC1 (17-AAG) (▪), EC82 (▴), EC86 (X) (EC82 and EC86 are purine based HSP90 inhibitors) or EC116 (an inactive structurally-related HSP90 inhibitor) (♦) for 24 hours at 37° C.

FIG. 6 compares by two-color flow cytometry the expression of ZAP70 in CLL B cells untreated (left panel) or treated with 300 nM EC1 (17-AAG) (right panel) for 24 hours at 37° C. The upper right quadrant were normal T-cells (CD3+, ZAP70+) the lower right quadrant were (CD3−, ZAP70+); the upper left quadrant is CD3+, ZAP70−; and the lower left quadrant is CD3−, ZAP70−.

FIG. 7 compares the % viability (expressed as 100%-% apoptotic cells) of ZAP70+ CCL B cells after treatment with EC1(17-AAG) (♦) or EC116 (inactive structurally-related HSP90 inhibitor) (▪).

FIG. 8 compares the % viability (expressed as 100%-% apoptotic cells) of ZAP70+ CCL B cells after treatment with 100 nM of EC1 (17-AAG) (▪) or EC116 (inactive structurally-related HSP90 inhibitor) (♦) The time taken to reach 50% cell mortality is approximately 48 hours after treating with 17-AAG.

FIG. 9 compares the viability (expressed as 100%-% apoptotic cells) of CCL B cells) from sixteen ZAP70+ patients and eleven ZAP70− patients after treatment with 100 nM EC1 (17-AAG) for 48 hours. ZAP70+ CLL B cells have an average % viability of 45.74+/−3.177%, whereas ZAP70− CLL B cells have an average % viability of 93+/−1.701%. The Students T-Test P-value of the difference in survival between the two populations was <0.0001.

### DETAILED DESCRIPTION OF THE INVENTION

The invention is directed to methods of treating an aggressive form of chronic lymphocytic leukemia (CLL) which is characterized by over expression of ZAP70, a protein kinase which normally found only in T cells, with HSP90 inhibitors. The method is based on the observation that ZAP70 co-immunoprecipates with HSP90, suggesting that it is an HSP90 client protein. The inventors further observed that in ZAP70 positive CLL samples, the majority of HSP90 present in the cytoplasm was in a complexed form, whereas in ZAP70 negative samples, most HSP90 was found to be uncomplexed. The inventors hypothesized that the abnormal overexpression and function in CLL B cells may depend on activated HSP90 and its level of expression may be down-regulated by using a specific HSP90 inhibitor, leading to induction of apoptosis of ZAP70 positive CLL B cells. It was found that the level of ZAP70 expression was decreased 30-40% in cells treated for 24 hours with HSP90 inhibitors at nanomolar concentrations.

### I. Definitions

The following terms have the following meanings and terms not specifically appearing below have their common customary meaning as used in the art:

The term “ZAP70 positive” and “ZAP70 negative” are based on a cutoff expression of 20% as measured by flow cytometry (FACSCalibur, BD Biosciences and Flow Jo software).

An “HSP90-inhibiting compound” or “HSP90-inhibitor” is one that disrupts the structure and/or function of an HSP90 chaperone protein and/or a protein that is dependent on HSP90. HSP90 proteins are highly conserved in nature (see, e.g., NCBI accession #'s P07900 and XM 004515 (human α and β HSP90, respectively), P11499 (mouse), AAB2369 (rat), P46633 (chinese hamster), JC1468 (chicken), AAF69019 (flesh fly), AAC21566 (zebrafish), AAD30275 (salmon), 002075 (pig), NP 015084 (yeast), and CAC29071 (frog)). Grp94 and Trap-1 are related molecules falling within the definition of an HSP90 as used herein. There are thus many different HSP90s, all with anticipated similar effect and inhibition capabilities. The HSP90 inhibitors of the invention may be specifically directed against an HSP90 of the specific host patient or may be identified based on reactivity against an HSP90 homolog from a different species or an HSP90 variant.

The term “ansamycin” is a broad term which characterizes compounds having an “ansa” structure which comprises any one of benzoquinone, benzohydroquinone, naphthoquinone or naphthohydroquinone moities bridged by a long chain. Compounds of the naphthoquinone or naphthohydroquinone class are exemplified by the clinically important agents rifampicin and rifamycin, respectively. Compounds of the benzoquinone class are exemplified by geldanamycin (including its synthetic derivatives 17-allylamino-17-demethoxygeldanamycin (17-AAG), 17-N,N-dimethylaminoethylamino-17-demethoxygeldanamycin (DMAG), dihydrogeldanamycin and herbamycin). The benzohydroquinone class is exemplified by macbecin. While the invention is illustrated using ansamycins, in particular 17-AAG, it should be understood that the novel method of treating CLL described herein applies to both the high melt and low melt forms of the compound, and its polymorphs, tautomers, enantiomers, pharmaceutically acceptable salts, and prodrugs. It should be further understand that the method further applies to many other ansamycins including, but not limited to, those exemplified in Examples 1-13 of the EXAMPLE section, such as geldanamycin, 17-N,N-dimethylaminoethylaminogeldanamycin, and polymorphs, tautomers, enantiomers, pharmaceutically acceptable salts, and prodrugs thereof. The structures of the numbered compounds are disclosed in the Summary section.

The term “pharmacologically active compound,” “active pharmaceutical ingredient” or “therapeutical ingredient” is synonymous with “drug” and means any compound that exerts, directly or indirectly, a biological effect, in vitro or in vivo when administered to cultured cells or to an organism.

A “prodrug” is a drug covalently bonded to a carrier wherein release of the drug occurs in vivo when the prodrug is administered to a mammalian subject. Prodrugs of the compounds of the present invention are prepared by modifying functional groups present in the compounds in such a way that the modifications are cleaved, either in routine manipulation or in vivo, to yield the desired compound. Prodrugs include compounds wherein hydroxy, amine, or sulfhydryl groups are bonded to any group that, when administered to a mammalian subject, is cleaved to form a free hydroxyl, amino, or sulfhydryl group, respectively. Examples of prodrugs include, but are not limited to, acetate, formate, or benzoate derivatives of alcohol or amine functional groups in the compounds of the present invention; phosphate esters, dimethylglycine esters, aminoalkylbenzyl esters, aminoalkyl esters or carboxyalkyl esters of alcohol or phenol functional groups in the compounds of the present invention; or the like. Prodrugs can impart multiple advantages for drug delivery, e.g., as explained in REMINGTON PHARMACEUTICAL SCIENCES, 20th Edition, Ch. 47, pp. 913-914.

“Pharmaceutically acceptable salts” include those derived from pharmaceutically acceptable inorganic and organic acids and bases. Examples of suitable acids include hydrochloric, hydrobromic, sulfuric, nitric, perchloric, fumaric, maleic, phosphoric, glycolic, gluconic, lactic, salicylic, succinic, toluene-p-sulfonic, tartaric, acetic, citric, methanesulfonic, formic, benzoic, malonic, naphthalene-2-sulfonic, benzenesulfonic, 1,2 ethanesulfonic acid (edisylate), galactosyl-d-gluconic acid and the like. Other acids, such as oxalic acid, while not themselves pharmaceutically acceptable, may be employed in the preparation of salts useful as intermediates in obtaining the compounds of this invention and their pharmaceutically acceptable acid addition salts. Salts derived from appropriate bases include alkali metal (e.g., sodium), alkaline earth metal (e.g., magnesium), ammonium and N-(C₁-C₄ alkyl)₄⁺ salts, and the like. Illustrative examples of some of these include sodium hydroxide, potassium hydroxide, choline hydroxide, sodium carbonate, and the like. Where the claims recite “a compound (e.g., compound ‘x’) or pharmaceutically acceptable salt thereof,” and only the compound is displayed, those claims are to be interpreted as embracing, in the alternative or conjunctive, a pharmaceutically acceptable salt or salts of such compound.

A “pharmaceutically effective amount” means an amount which is capable of providing a therapeutic or prophylactic effect. The specific dose of compound administered according to this invention to obtain therapeutic and/or prophylactic effect will, of course, be determined by the. particular circumstances surrounding the case, including, for example, the specific compound administered, the route of administration, the condition being treated, and the individual being treated. A typical daily dose (administered in single or divided doses) will contain a dosage level of from about 0.01 mg/kg to about 100 and more preferably 50 mg/kg of body weight of an active compound of this invention. Preferred daily doses generally will be from about 0.05 mg/kg to about 20 mg/kg and ideally from about 0.1 mg/kg to about 10 mg/kg.

The preferred therapeutic effect is the inhibition, to some extent, of the growth of cells characteristic of the disorder treated. A therapeutic effect will also normally, but need not, relieve to some extent one or more of the symptoms associated with the disorder.

The term “IC₅₀” is defined as the concentration of an HSP90 inhibitor required to achieve killing of 50% of the cells of a population, or of a particular cell type, e.g., cancerous versus noncancerous cells within a greater cell population. The IC₅₀ is preferably, although not necessarily, greater for normal cells than for cells exhibiting a proliferative disorder.

A “physiologically acceptable carrier” refers to a carrier or diluent that does not cause significant irritation to an organism and does not abrogate the biological activity and properties of the administered compound. Depending on the formulation, the diluent can be a solid such as calcium carbonate, sodium carbonate, lactose, calcium phosphate or sodium phosphate, or a liquid, such as water or oils.

An “excipient” refers to a non-toxic pharmaceutically acceptable substance added to a pharmacological composition to facilitate the processing, administration and pharmaceutics properties of a compound. Excipients may include but are not limited to, fillers, diluents, glidants, lubricants, disintegrants, binders, solubilizers, stabilizers/bulking agents, and various functional and non-functional coatings.

The term “about” means including and exceeding up to 15% the specific endpoint(s) designated. Thus the range is broadened.

The term “optionally” denotes that the step or component following the term may but need not be a part of the method or formulation.

### II. Preparation of Ansamycins

Ansamycins according to this invention may be synthetic, naturally-occurring, or a combination of the two, i.e., “semi-synthetic,” and may include dimers and conjugated variant and prodrug forms. Some exemplary benzoquinone ansamycins useful in the various embodiments of the invention and their methods of preparation include but are not limited to those described, e.g., in U.S. Pat. No. 3,595,955 (describing the preparation of geldanamycin), No. 4,261,989, No. 5,387,584, and No. 5,932,566 and those described in the “EXAMPLE” section (Examples 1-12), below. Geldanamycin is also commercially available, e.g., from CN Biosciences, an Affiliate of Merck KGaA, Darmstadt, Germany, headquartered in San Diego, Calif., USA (cat. no. 345805). 17-N,N-dimethylaminoethylamino-17-desmethoxy-geldanamycin (DMAG) is commercially available from EMD/Calbiochem. The biochemical purification of the geldanamycin derivative, 4,5-dihydrogeldanamycin and its hydroquinone from cultures of Streptomyces hygroscopicus (ATCC 55256) are described in WO 93/14215 (Cullen et al.): An alternative method of synthesis for 4,5-dihydrogeldanamycin by catalytic hydrogenation of geldanamycin is also known. See e.g., “Progress in the Chemistry of Organic Natural Products,” Chemistry of the Ansamycin Antibiotics, 1976 33:278. Other ansamycins that can be used in connection with various embodiments of the invention are described in the literature cited in the “Background” section and also in the “Summary” section, above.

17-AAG may be prepared from geldanamycin by reacting with allyamine in dry THF under a nitrogen atmosphere. The crude product may be purified by slurrying in H₂O:EtOH (90:10), and the washed crystals obtained have a melting point of 206-212° C. by capillary melting point technique. A second product of 17-AAG can be obtained by dissolving and recrystallizing the crude product from 2-propyl alcohol (isopropanol). This second 17-AAG product has a melting point between 147-153° C. by capillary melting point technique. The two 17-AAG products are designated as the low melt form and high melt form. The stability of the low melt form may be tested by slurring the crystals in the solvent (H₂O:EtOH (90:10)) from which the high melt form was purified; no conversion to the high melt form was observed. See Examples 1-2 for details of the preparation of the two polymorphic forms of 17-AAG.

### III. Characterization and Evaluation of the Effectiveness of Down Regulating ZAP70 by Inhibition of HSP90

A. Determining ZAP70 Levels in Cell Lysates

Many different types of methods are known in the art for determining protein concentrations and measuring or predicting the level of proteins within cells and in fluid samples. Indirect techniques include nucleic acid hybridization and amplification using, e.g., polymerase chain reaction (PCR). These techniques are known to the person of skill and are discussed, e.g., in Sambrook, Fritsch & Maniatis, MOLECULAR CLONING: A LABORATORY MANUAl, Second Edition (1989) Cold Spring Harbor Laboratory Press, Cold Spring Harbor, N.Y., Ausubel, et al., CURRENT PROTOCOLS IN MOLECULAR BIOLOGY, John Wiley & Sons, NY, 1994. The concentration of ZAP70 may be determined by immunoassay techniques such as immunoblotting, radioimmunoassay, immunofluorescence, western blotting, immunoprecipitation, enzyme-linked immunosorbant assays (ELISA), and derivative techniques that make use of antibodies directed against ZAP70, and flow cytometry. A convenient and quantitative method of determining ZAP70 expression is FACS (fluorescence-activated cell sorter) (FACSCalibur, BD Biosciences and Flow-Jo software, version 2.7.4 (Tree Star)), a version of flow cytometry, which is described in a recently published study by Rassenti et al. supra and the disclosure of which is incorporated herein by reference. In the Rassenti study, the blood cells were stained with CD19-specific and CD3-specific monoclonal antibodies conjugated with allophycocyanin and phycoerythrin, respectively (Pharmingen) and also with anti-ZAP70 monoclonal antibody that had been conjugated to Alexa-488 dye (Becton Dickenson). Other dye systems may also be used. Lymphocytes were gated on the basis of their forward-angle light scatter and side-angle light scatter, and blood mononuclear cells from a healthy donor can be used to establish the initial gate. The expression of ZAP70 was measured by calculating the percentage of CD19+CD3− cells that was above this gating threshold. ZAP70 positive and ZAP70 negative can be based on a cutoff expression, e.g., as expression of ZAP70 detected by flow cytometry in more than 20% of leukemia cells.

B. Determining the Binding Affinity of HSP90 Ligands to HSP90

A variety of isotopic and nonisotopic methods, e.g., colorimetric, enzymatic, and densitometric, afford sufficient sensitivity to evaluate the binding affinity of an inhibitor to a target protein. These methods are generally known in the art and can be used in the context of this invention.

The binding affinity of HSP90 ligands to HSP90 can also be measured by the competitive binding assay described in Kamal et al., Nature 2003, 425:407-410, the disclosure of which is incorporated herein by reference. The binding affinity of the ligand is measured by its ability to inhibit the binding of geldanamycin, a known inhibitor of HSP90. The cell containing the HSP90 is first lysed in lysis buffer. The lysates were incubated with or without 17-AAG and then incubated with biotin-GM linked to BioMag™ streptavidin magnetic beads (Qiagen). The bound samples and the unbound supernatant can be separately collected and analyzed on SDS protein gels, and blotted using an HSP90 antibody (StressGen, SPA-830). The bands in the Western blots may be quantitated using the Bio-rad Fluor-S MultiImager, and the % inhibition of binding of HSP90 to the biotin-GM was calculated. The IC₅₀ is the concentration of HSP90 ligand needed to cause half-maximal inhibition of binding.

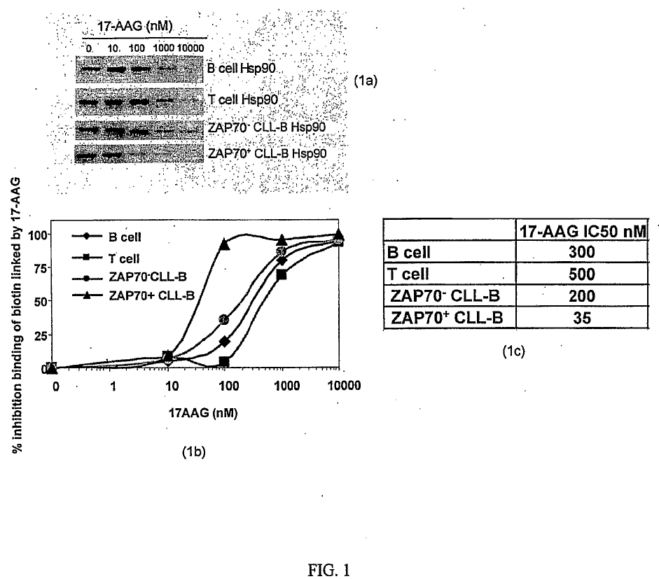

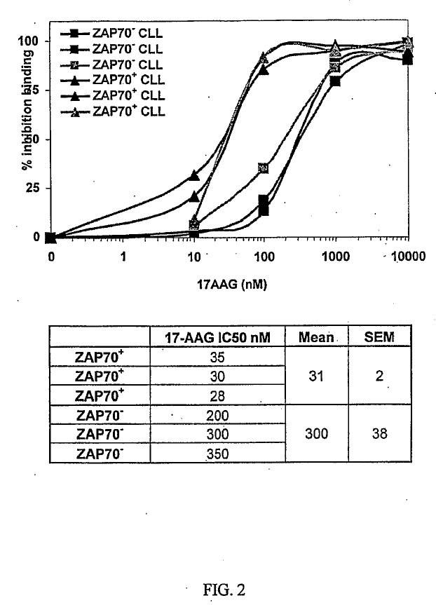

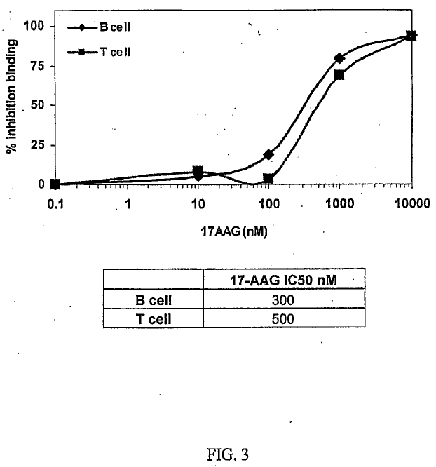

FIGS. 1-3 show the competitive binding of 17-AAG against a biotinylated geldanamycin probe (biotin-GM) for HSP90 in lysates of B cells, T cells, ZAP70 positive CLL B cells and ZAP70 negative CLL B cells. The Western blot bands show that inhibition of binding of HSP90 to the biotin-GM decreases with increasing concentration of 17-AAG (1a.). The results are quantitated and plotted in % inhibition of binding of HSP90 to the biotin-GM vs. 17-AAG concentration in nM (1b). The figures show that the inhibition of binding is higher for HSP90 isolated from ZAP70+ CLL B cells. The calculated IC₅₀ shows that 17 AAG has an approximately 10× higher binding affinity for HSP90 isolated from ZAP70+ CLL B cells compared to ZAP70− CLL B cells and to normal B cells.

C. Determining the Association of the Proteins by Co-immunoprecipitation

The inter-association of various proteins can be determined by co-immunoprecipation experiments using antibodies specific for the proteins of interest. These methods are well known in the art. See, Goldsby R. A. et al., KIRBY IMMUNOLOGY, 4th Edition, W.H. Freeman and Company, 2000.

To determine whether ZAP70 is a client protein of HSP90 and whether HSP90 in the ZAP70+ CLL B cells is present in multi-chaperone complexes, a set of four co-immunopreciptation experiments were performed. MCF-7 breast carcinoma cells, primary isolated of ZAP70+ and ZAP70− chronic lymphocytic leukemia (CLL B) cells and normal T and B cells were lysed and incubated with pre-blocked protein-A Sepharose beads (Zymed) with antibodies specific for the protein of interest. The bound and unbound fractions can be separately collected and analyzed by SDS-PAGE and Western blots using the indicated antibodies.

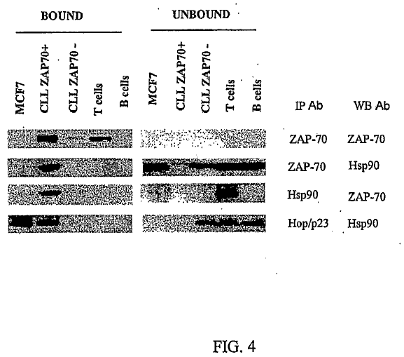

FIG. 4 shows the immunoblot of the co-immunoprecipitation experiment. The antibodies used in each step of the experiment were indicated. IP Ab denotes the antibody used during immunoprecipation. WB Ab denotes the antibody used during Western Blot. P23 and HOP are essential components of the two known multi-chaperone HSP90 complexes. The first gels demonstrate that ZAP70 is expressed in ZAP70+ CLL B cells and normal T cells, but not in ZAP70− CLL B cells or normal B cells. The second gels show that ZAP70 is physically associated with HSP90 in ZAP70+ CLL B cells, but not in any of the other cell types, including normal T cells. The third gels confirm the previous finding by reversing the co-immunoprecipitation. The fourth gels show that HSP90 in MCF-7 cells (the positive control, see Kamal et al., Nature, 2003, 425:407-410) and in ZAP70+ CLL B cells is in activated state (multichaperone complexes with HOP and p23), whereas HSP90 in ZAP70− CLL B cells or normal T or B cells is in the latent resting state (not associated with HOP or p23).

### IV. Characterization and Evaluation of the Effectiveness of Inhibition of ZAP70

The downstream effect on ZAP70 by inhibition of HSP90 can be directly measured by the amount of ZAP70 expression or by determining the viability of the cells after treatment with selected HSP90 inhibitors.

Primary isolates of B-cell chronic lymphocytic leukemia cells from an individual ZAP70+ patient were treated with EC1 (17-AAG), EC82 or EC86 (two purine based known HSP90 inhibitors) or EC 116 (an inactive structurally-related HSP90 inhibitor) for 24 hours at 37° C. Levels of ZAP70 protein expression were measured by indirect immunofluorescence of permeabilized cells with specific anti-ZAP70 antibodies and FACS analysis.

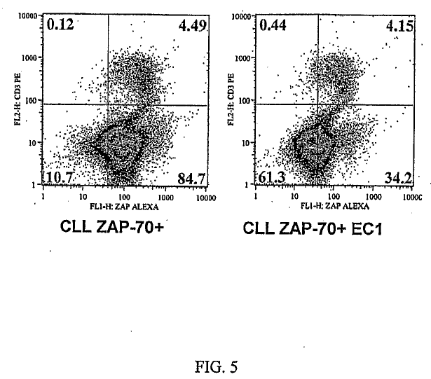

FIG. 5 shows that all three active HSP90 inhibitors dose-dependently induced degradation of ZAP70, confirming that ZAP70 is an HSP90-dependent client protein, as was indicated by the physical association demonstrated in the co-immunoprecipitation experiments (FIG. 4). Additionally, the fact that three structurally-unrelated HSP90 inhibitors produced the same effect strongly implicates HSP90 as an essential protein for the stability of ZAP70 in CLL B cells.

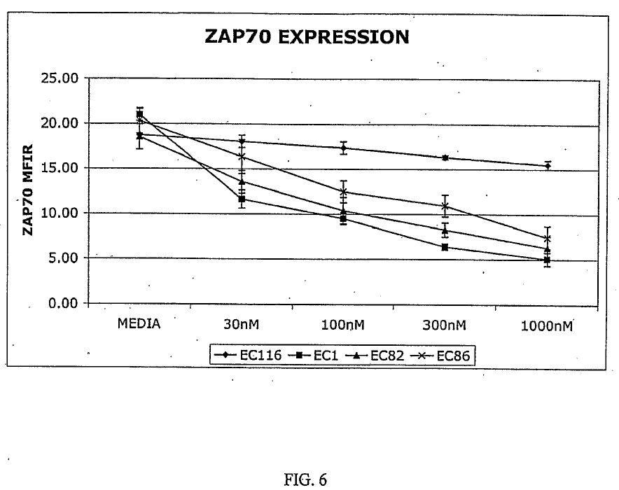

Primary isolates of white blood cells from an individual ZAP70+ B-cell chronic lymphocytic leukemia patient were left untreated (left panel) or treated with 300 nM EC1 (17-AAG) for 24 hours at 37° C. (right panel). Levels of ZAP70 protein expression were measured by two color indirect immunofluorescence with specific anti-CD3 antibodies conjugated to phycoerytirin and anti-ZAP70 antibodies conjugated to Alexa-488 dye and analyzed by flow cytometry. CD3 is a specific marker of T cells. FIG. 6 compares the expression of ZAP70 in untreated CLL-B cells untreated (left panel) or treated (300 nM 17-AAG) (right panel) cells. As shown in the untreated cells (left panel), approximately 5% of the cells were normal T-cells (CD3+, ZAP70+, upper right quadrant) and ˜85% of the cells were ZAP70+ CLL B cells (CD3−, ZAP70+, lower right quadrant). EC1 (17-AAG) induced degradation of ZAP70 in the B-CLL cells (% positive cells 85%→34%), but not in the normal T-cells (% positive cells 4.5%→4.2%), as predicted from the physical association demonstrated in B-CLL cells, but not in the normal T-cells in the co-immunoprecipitation experiments (FIG. 4). The finding that HSP90 inhibitors induce ZAP70 degradation in B-CLL cells but not normal T-cells indicates that such drugs would have a more specific antileukemic activity than ZAP70 kinase inhibitors. This is important because B-CLL patients are chronically immunosuppressed by their disease, so avoidance of effects on normal T-cell function performed by ZAP70 is clearly beneficial.

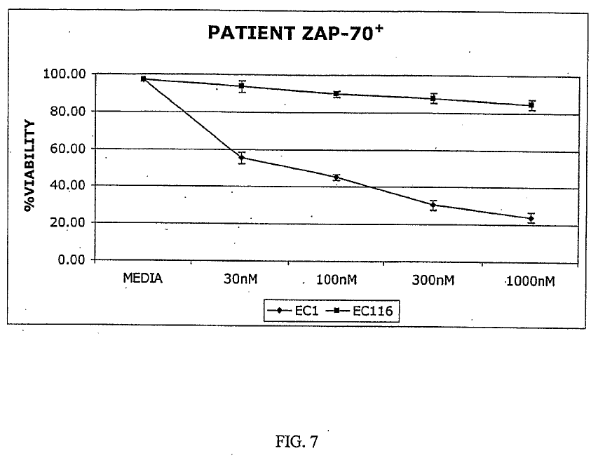

Primary isolates of CLL B cells from an individual ZAP70+ patient were treated with EC1 (17-AAG) or EC116 (inactive structurally-related HSP90 inhibitor) for 48 hours at 37° C. Apoptotic cells were identified by a standard protocol using the mitochondrial vital dye DiOC6 and propidium iodide staining. The % viability is expressed as 100%-% apoptotic cells. FIG. 7 shows compares the % viability of ZAP70+ chronic lymphocytic leukemia B cells after treating with EC1 (17-AAG) (♦) or EC116 (inactive structurally-related HSP90 inhibitor) (▪). It is obviously show that ZAP70+ CLL B cells were readily killed by 17-AAG, with a 50% inhibitory concentration (IC₅₀) of approximately 80 nM.

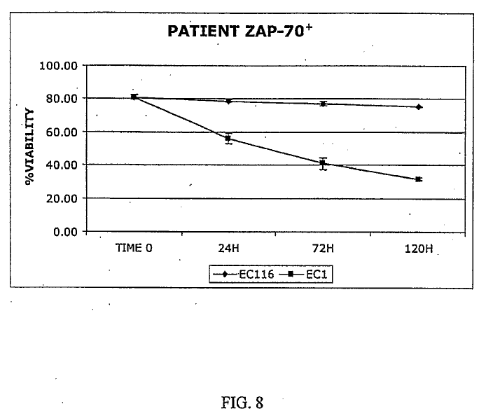

A similar experiment was performed to measure the rate at which ZAP70+ CLL B cells succumb. Primary isolates of CLL B cells from an individual ZAP70+ patient were treated with 100 nM EC1 (17-AAG) or EC116 (inactive structurally-related HSP90 inhibitor) for varying times at 37° C. Apoptotic cells were identified by a standard protocol using the mitochondrial vital dye DiOC6 and propidium iodide staining. The % viability is expressed as 100%-% apoptotic cells. FIG. 8 compares the % viability of ZAP70+ chronic lymphocytic leukemia B cells after treating with 100 nM of EC1 (17-AAG) (▪) or EC 116 (inactive structurally-related HSP90 inhibitor) (♦). The result indicates that ZAP70+ tumor cells were rapidly killed by 17-AAG, with a 50% of the cells succumbing in approximately 48 hours.

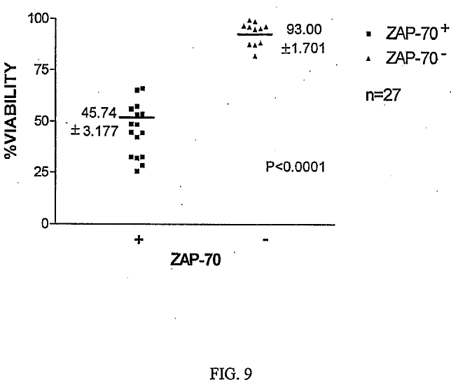

Primary isolates of CCL B cells from sixteen ZAP70+ patients and eleven ZAP70− patients were treated with 100 nM EC1 (17-AAG) for 48 hours at 37° C. Apoptotic cells were identified by a standard protocol using the mitochondrial vital dye DiOC6 and propidium iodide staining. The % viability is expressed as 100%-% apoptotic cells. FIG. 9 compares the viability of CLL B cells from the sixteen ZAP70+ patients and the eleven ZAP70− patients after treatment with 100 nM EC1 (17-AAG) for 48 hours. ZAP70+ CLL B cells have an average % viability of 45.74+/−3.177%, whereas ZAP70− B CLL cells have an average % viability of 93+/−1.701%. The Students T-Test P-value of the difference in survival between the two populations was <0.0001 which is highly statistically significant.

### V. Formulation and Administration of Pharmaceutical Compositions

Geldanamycin may be prepared according to U.S. Pat. No. 3,595,955 using the subculture of Streptomyces hygroscopicus that is on deposit with the U.S. Department of Agriculture, Northern Utilization and Research Division, Agricultural Research, Peoria, Ill., USA, accession number NRRL 3602. Numerous derivatives of this compound may be fashioned as specified in U.S. Pat. Nos. 4,261,989, 5,387,584, and 5,932,566, according to standard techniques.

Those of ordinary skill in the art are familiar with formulation and administration techniques that can be employed in use of the invention, e.g. as discussed in Goodman and Gilman's, THE PHARMACOLOGICAL BASIS OF THERAPEUTICS, current edition; Pergamon Press; and REMINGTON'S PHARMACEUTICAL SCIENCES (current edition.) Mack Publishing Co., Easton, Pa.

The compounds utilized in the methods of the instant invention may be administered either alone or in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

For example, the therapeutic or pharmaceutical compositions of the invention can be administered locally to the area in need of treatment. This may be achieved by, for example, but not limited to, local infusion during surgery, topical application, e.g., cream, ointment, injection, catheter, or implant, said implant made, e.g., out of a porous, nonporous, or gelatinous material, including membranes, such as sialastic membranes, or fibers. The administration can also be by direct injection at the site (or former site) of a tumor or neoplastic or pre-neoplastic tissue.

Still further, the therapeutic or pharmaceutical composition can be delivered in a vesicle, e.g., a liposome (see, for example, Langer, Science, 1990, 249:1527-1533; Treat et al., Liposomes in the Therapy of Infectious Disease and Cancer, 1989, LopezBernstein and Fidler (eds.), Liss, N.Y., pp. 353-365).

The pharmaceutical compositions used in the methods of the present invention can be delivered in a controlled release system. In one embodiment, a pump may be used (see, Sefton, CRC Crit. Ref. Biomed. Eng. 1987, 14:201; Buchwald, et al., Surgery, 1980, 88:507; Saudek et al., N. Engl. J. Med., 1989, 321:574). Additionally, a controlled release system can be placed in proximity of the therapeutic target. (see, Goodson, Medical Applications of Controlled Release, 1984, Vol. 2, pp. 115-138).

The pharmaceutical compositions used in the methods of the instant invention can contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose or cellulose acetate butyrate, may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions used in the methods of the instant invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution is then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleaginous suspension for intramuscular and subcutaneous administration. This suspension may be formulated according to the known art using those suitable dispersing or wetting agents and suspending agents which have been mentioned above. The sterile injectable preparation may. also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The HSP90 inhibitors used in the methods of the present invention may also be administered in the form of suppositories for rectal administration of the drug. These compositions can be prepared by mixing the inhibitors with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing an HSP90 inhibitor can be used. As used herein, topical application can include mouth washes and gargles.

The compounds used in the methods of the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The methods and compounds of the instant invention may also be used in conjunction with other well known therapeutic agents that are selected for their particular usefulness against the condition that is being treated. For example, the instant compounds may be useful in combination with known anti-cancer and cytotoxic agents.

Further, the instant methods and compounds may also be useful in combination with other inhibitors of parts of the signaling pathway that links cell surface growth factor receptors to nuclear signals initiating cellular proliferation.

The methods of the present invention may also be useful with other agents that inhibit angiogenesis and thereby inhibit the growth and invasiveness of tumor cells, including, but not limited to VEGF receptor inhibitors, including ribozymes and antisense targeted to VEGF receptors, angiostatin and endostatin.

Examples of antineoplastic agents, which can be used in combination with the methods of the present invention include, in general, alkylating agents, anti-metabolites; epidophyllotoxin; an antineoplastic enzyme; a topoisomerase inhibitor; procarbazine; mitoxantrone; platinum coordination complexes; biological response modifiers and growth inhibitors; hormonal/antihormonal therapeutic agents and haematopoietic growth factors.

Example classes of antineoplastic agents include, for example, the anthracycline family of drugs, the vinca drugs, the mitomycins, the bleomycins, the cytotoxic nucleosides, the epothilones, discodermolide, the pteridine family of drugs, diynenes and the podophyllotoxins. Particularly useful members of those classes include, for example, carminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

When a HSP90 inhibitor used in the methods of the present invention is administered into a human subject, the daily dosage will normally be determined by the prescribing physician with the dosage generally varying according to the age, weight, and response of the individual patient, as well as the severity of the patient's symptoms.

In one exemplary application, a suitable amount of a HSP90 inhibitor is administered to a mammal undergoing treatment for cancer, for example, breast cancer. Administration occurs in an amount of each type of inhibitor of between about 0.1 mg/kg of body weight to about 60 mg/kg of body weight per day, preferably of between 0.5 mg/kg of body weight to about 40 mg/kg of body weight per day. A particular therapeutic dosage that comprises the instant composition includes from about 0.01 mg to about 1000 mg of a HSP90 inhibitor. Preferably, the dosage comprises from about 1 mg to about 1000 mg of a HSP90 inhibitor.

Preferably, the pharmaceutical preparation is in unit dosage form. In such form, the preparation is subdivided into unit doses containing appropriate quantities of the active component, e.g., an effective amount to achieve the desired purpose.

The quantity of active compound in a unit dose of preparation may be varied or adjusted from about 0.1 mg to 1000 mg, preferably from about 1 mg to 300 mg, more preferably 10 mg to 200 mg, according to the particular application.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The amount and frequency of administration of the HSP90 inhibitors used in the methods of the present invention and if applicable other chemotherapeutic agents and/or radiation therapy will be regulated according to the judgment of the attending clinician (physician) considering such factors as age, condition and size of the patient as well as severity of the disease being treated.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the patient, and in view of the observed responses of the disease to the administered therapeutic agents.

Also, in general, the HSP90 inhibitor and the chemotherapeutic agent do not have to be administered in the same pharmaceutical composition, and may, because of different physical and chemical characteristics, have to be administered by different routes. For example, the HSP90 inhibitor may be administered orally to generate and maintain good blood levels thereof, while the chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of HSP90 inhibitor, and chemotherapeutic agent and/or radiation will depend upon the diagnosis of the attending physicians and their judgment of the condition of the patient and the appropriate treatment protocol.

The HSP90 inhibitor, and chemotherapeutic agent and/or radiation may be administered concurrently (e.g., simultaneously, essentially simultaneously or within the same treatment protocol) or sequentially, depending upon the nature of the proliferative disease, the condition of the patient, and the actual choice of chemotherapeutic agent and/or radiation to be administered in conjunction (i.e., within a single treatment protocol) with the HSP90 inhibitor.

If the HSP90 inhibitor and the chemotherapeutic agent and/or radiation are not administered simultaneously or essentially simultaneously, then the initial order of administration of the HSP90 inhibitor and the chemotherapeutic agent and/or radiation may not be important. Thus, the HSP90 inhibitor may be administered first followed by the administration of the chemotherapeutic agent and/or radiation; or the chemotherapeutic agent and/or radiation may be administered first followed by the administration of the HSP90 inhibitor. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the chemotherapeutic agent and/or radiation may be administered first, especially if it is a cytotoxic agent, and then the treatment continued with the administration of the HSP90 inhibitor followed, where determined advantageous, by the administration of the chemotherapeutic agent and/or radiation, and so on until the treatment protocol is complete.

Thus, in accordance with experience and knowledge, the practicing physician can modify each protocol for the administration of a component (therapeutic agent-i.e., HSP90 inhibitor, chemotherapeutic agent or radiation) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

### VI. Method of Using the Formulations

A. Dose Range

A phase I pharmacologic study of 17-AAG in adult patients with advanced solid tumors determined a maximum tolerated dose (MTD) of 40 mg/m² when administered daily by 1-hour infusion for 5 days every three weeks. (Wilson et al., Am. Soc. Clin. Oncol., abstract, “Phase I Pharmacologic Study of 17-(Allylamino)-17-Demethoxygeldanamycin (AAG) in Adult Patients with Advanced Solid Tumors” 2001.) In this study, mean±SD values for terminal half-life, clearance and steady-state volume were determined to be 2.5±0.5 hours, 41.0±13.5 L/hour, and 86.6±34.6 L/m², respectively. Plasma Cmax levels were determined to be 1860±660 nM and 3170±1310 nM at 40 and 56 mg/m². Using these values as guidance, it is anticipated that the range of useful patient dosages for formulations of the present invention will include between about 0.40 mg/m² and 4000 mg/m² of active ingredient, where m² represents surface area. Standard algorithms exist to convert mg/m² to mg of drug/kg patient bodyweight.

### EXAMPLES

The following examples are offered by way of illustration only, and all drugs, components, molar ratios, concentrations, pH and steps included therein are not intended to be limiting of the invention unless specifically recited in the claims. Compound preparations of Examples 1-12 are reproduced appropriately below, from commonly owned U.S. Provisional Application Ser. Nos. 60/371,668 and 60/478,430, and International Application PCT/US03/10533, entitled NOVEL ANSAMYCIN FORMULATIONS AND METHODS FOR PRODUCING AND USING SAME, filed Apr. 4, 2003, and International Application PCT/US 03/1053, entitled DRUG FORMULATIONS HAVING LONG AND MEDIUM CHAIN TRIGLYCERIDES, filed Oct. 4, 2003, and to which this application claims priority.

### Example 1

### Preparation of 17-AAG

To 45.0 g (80.4 mmol) of geldanamycin in 1.45 L of dry THF in a dry 2 L flask was added drop-wise over 30 minutes 36.0 mL (470 mmol) of allyl amine in 50 mL of dry THF. The reaction mixture was stirred at room temperature under nitrogen for 4 hr at which time TLC analysis indicated the reaction was complete [(GDM: bright yellow: Rf=0.40; (5% MeOH-95% CHCl₃); 17-AAG: purple: Rf=0.42 (5% MeOH-95% CHCl₃)]. The solvent was removed by rotary evaporation and the crude material was slurried in 420 mL of H₂O:EtOH (90:10) at 25° C., filtered and dried at 45° C. for 8 hr to give 40.9 g (66.4 mmol) of 17-AAG as purple crystals (82.6% yield, >98% pure by HPLC monitored at 254 nm). MP 206-212° C. ¹H NMR and HPLC are consistent with the desired product.

### Example 2

### Preparation of a Low Melt Form of 17-AAG

The crude 17-AAG from Example 1 is dissolved in 800 mL of 2-propyl alcohol (isopropanol) at 80° C. and then cooled to room temperature. Filtration followed by drying at 45° C. for 8 hr gives 44.6 g (72.36 mmol) of 17-AAG as purple crystals (90% yield, >99% pure by HPLC monitored at 254 nm). MP=147-153° C. ¹H NMR and HPLC are consistent with the desired product.

### Example 3

### Solvant Stability of a Low Melt Form of 17-AAG

The 17-AAG product from Example 2 in 400 mL of H₂O:EtOH (90:10) at 25° C., filter and dry at 45° C. for 8 hr to give 42.4 g (68.6 mmol) of 17-AAG as purple crystals (95% yield, >99% pure by HPLC monitored at 254 nm). MP=147-175° C. ¹H NMR and HPLC are consistent with the desired product.

### Example 4

### Preparation of Compound 237: A Dimer

3,3-diamino-dipropylamine (1.32 g, 9.1 mmol) was added dropwise to a solution of geldanamycin (10 g, 17.83 mmol) in DMSO (200 mL) in a flame-dried flask under N₂ and stirred at room temperature. The reaction mixture was diluted with water after 12 hours. A precipitate was formed and filtered to give the crude product. The crude product was chromatographed by silica chromatography (5% CH₃OH/CH₂Cl₂) to afford the desired dimer as a purple solid. The pure purple product was obtained after flash chromatography (silica gel); yield: 93%; mp 165° C.; ¹H NMR (CDC₁₃) 0.97 (d, J=6.6 Hz, 6H, 2CH₃), 1.0 (d, J=6.6 Hz, 6H, 2CH₃), 1.72 (m, 4H, 2 CH₂), 1.78 (m, 4H, 2CH₂), 1.80 (s, 6H, 2CH₃), 1.85 (m, 2H, 2CH), 2.0 (s, 6H, 2CH₃), 2.4 (dd, J=11 Hz, 2H, 2CH), 2.67 (d, J=15 Hz, 2H, 2CH), 2.63 (t, J=10 HZ, 2H, 2CH), 2.78 (t, J=6.5 Hz, 4H, 2CH₂), 3.26 (s, 6H, 20CH₃), 3.38 (s, 6H, 20CH₃), 3.40 (m, 2H, 2CH), 3.60 (m, 4H, 2CH₂), 3.75 (m, 2H, 2CH), 4.60 (d, J=10 Hz, 2H, 2CH), 4.65 (Bs, 2H, 20H), 4.80 (Bs, 4H, 2NH2), 5.19 (s, 2H, 2CH), 5.83 (t, J=15 Hz, 2H, 2CH═), 5.89 (d, J=10 Hz, 2H, 2CH═), 6.58 (t, J=15 Hz, 2H, 2CH═), 6.94 (d, J=10 Hz, 2H, 2CH═), 7.17 (m, 2H, 2NH), 7.24 (s, 2H, 2CH═), 9.20 (s, 2H, 2N—H); MS (m/z) 1189 (M+H).

The corresponding HCl salt was prepared by the following method: an HCl solution in EtOH (5 ml, 0.12 3N) was added to a solution of compound #237 (1 g as prepared above) in THF (15 ml) and EtOH (50 ml) at room temperature. The reaction mixture was stirred for 10 min. The salt was precipitated, filtered and washed with a large amount of EtOH and dried in vacuo. Alternatively, a “mesylate” salt can be formed using methanesulfonic acid instead of HCl.

### Example 5

### Preparation of Compound 914

To geldanamycin (500 mg, 0.89 mmol) in 10 mL of dioxane was added selenium (IV) dioxide (198 mg, 1.78 mmol) at room temperature. The reaction mixture was heated to 100° C. and stirred for 3 hours. After cooling to room temperature, the solution was diluted with ethyl acetate, washed with water and brine, dried over magnesium sulfate, filtered and evaporated in vacuo. The final pure yellow product was obtained after column chromatography (silica gel); yield: 75%; ¹H NMR (CDCl₃) δ 0.97(d, J=7.OHz, 3H, CH3),1.01(d, J=7.OHz, 3H, CH₃), 1.75(m, 3H, CH₂+CH), 2.04(s, 3H, CH₃), 2.41(d, J=9.9Hz, 1H, CH₂), 2.53(t, J=9.9 Hz, 1H, CH₂), 2.95(m, 1H, CH), 3.30(m, 2H, CH+OH), 3.34(s, 6H, 2CH₃), 3.55(m, 1H, CH), 4.09(m, 1H, CH₂), 4.15(s, 3H, CH₃), 4.25(m, 1H, CH₂), 4.41(d, J=9. 8Hz, 1H, CH), 4.80(bs, 2H, CONH₂), 5.32(s, 1H, CH), 5.88(t, J=10.4 Hz, 1H, CH═), 6.04(d, J=9.7 Hz, 1H, CH═), 6.65(t, J=11.5 Hz, 1H, CH═), 6.95(d, J=11.5 Hz, 1H, CH═), 7.32(s, 1H, CH—Ar), 8.69(s, 1H, NH); MS (m/z) 575.6 (M−1).

### Example 6

### Preparation of Compound 967

To compound #914 (50 mg, 0.05 mmol) in 3 mL of THF was added allylamine (3.5 mg, 0.06 mmol). The reaction mixture was stirred at room temperature for 24 hours. The solvent was removed by rotary evaporation. The pure purple product was obtained after column chromatography (silica gel); yield: 90%; ¹H NMR (CDCl₃) δ0.89(d, J=6.6 Hz, 3H, CH₃), 1.03 (d, J=6.9 Hz, 3H, CH₃), 1.78(m, 1H, CH), 1.82(m, 2H, CH₂), 2.04 (s, 3H, CH₃), 2.37(dd, J=13.7 Hz, 1H, CH₂), 2.65(d, J=13.7 Hz, 1H, CH₂), 2.90(m, 1H, CH), 3.33(s, 3H, CH₃), 3.34(s, 3H, CH₃), 3.45(m, 2H, CH+OH), 3.58(m, 1H, CH), 4.14(m, 3H, CH₂+CH₂), 4.16(m, 1H, CH₂), 4.42(s, 1H, OH), 4.43(d, J=10 Hz, 1H, CH), 4.75(bs, 2H, CONH₂), 5.33(m, 2H, CH₂═), 5.35(s, 1H, CH), 5.91(m, 2H, CH═+CH═), 6.09(d, J=9.9 Hz, 1H, CH═), 6.46(t, J=5.8 Hz, 1H, NH), 6.66(t, J=11.6 Hz, 1H, CH═), 6.97(d, J=11.6 Hz, 1H, CH═), 7.30(s, 1H, CH), 9.15(s, 1H, NH).

### Example 7

### Preparation of Compound 956

Compound #956 was prepared by the same method described for compound #967 except that azetidine was used instead of allylamine. The final pure purple product was obtained after column chromatography (silica gel); yield: 89%; ¹H NMR (CDCl₃) δ 0.99 (d, J=6.8 Hz, 3H, CH₃), 1.04 (d, J=6.8 Hz, 3H, CH₃), 1.77 (m, 1H, CH), 1.80 (m, 2H, CH₂), 2.06 (s, 3H, CH₃), 2.26 (m, 1H, CH₂), 2.50(m, 2H, CH₂), 2.67 (d, 1H, CH₂), 2.90 (m, 1H, CH), 3.34 (s, 3H, CH₃), 3.36 (s, 3H, CH₃), 3.48 (m,2H, OH+CH), 3.60 (t, J=6.8 Hz, 1H, CH), 4.11 (dd, J=12 Hz, J=4.5 Hz, 1H, CH₂), 4.30 (dd, J=12 Hz, J=4.5 Hz, 1H, CH₂), 4.45 (d, J=10.0 Hz, 1H, CH), 4.72 (m, 5H, 2CH₂+OH), 4.78 (bs, 2H, NH₂), 5.37 (s, 1H, CH), 5.89 (t, J=10.5 Hz, 1 H, CH═), 6.10 (d, J=10 Hz, 1 H, CH═), 6.66 (t, J=12 Hz, 1 H, CH═), 7.00 (d, J=12 Hz, 1H, CH═), 7.17 (s, 1H, CH═), 9.20 (s, 1H, CONH); MS(m/z) 602 (M+1).

### Example 8

### Preparation of Compound 529

A solution of 17-aminogeldanamycin (1 mmol) in EtOAc was treated with Na₂S₂O₄ (0.1 M, 300 ml) at RT. After 2 h, the aqueous layer was extracted twice with EtOAc and the combined organic layers were dried over Na₂SO₄, concentrated under reduce pressure to give 18,21-dihydro-17-aminogeldanamycin as a yellow solid. This solid was dissolved in anhydrous THF and transferred via cannula to a mixture of picolinoyl chloride (1.1 mmol) and MS4A (1.2 g). Two hours later, EtN(i-Pr)₂ (2.5 mmol) was further added to the reaction mixture. After overnight stirring, the reaction mixture was filtered and concentrated under reduce pressure. Water was then added to the residue, which was extracted with EtOAc three times; the combined organic layers were dried over Na₂SO₄ and concentrated under reduce pressure to give the crude product which was purified by flash chromatography to give 17-picolinoyl-aminogeldanamycin, Compound 529, as a yellow solid. Rf=0.52 in 80:15:5 CH₂Cl₂: EtOAc: MeOH. Mp=195-197° C. ¹H NMR (CDCl₃) δ 0.91 (d, 3H), 0.96 (d, 3H), 1.71-1.73 (m, 2H), 1.75-1.79 (m, 4H), 2.04 (s, 3H), 2.70-2.72 (m, 2H), 2.74-2.80 (m, 1H), 3.33-3.35 (m, 7H), 3.46-3.49 (m, 1H), 4.33 (d, 1H), 5.18 (s, 1H), 5.77 (d, 1H), 5.91 (t, 1H), 6.57 (t, 1H), 6.94 (d, 1H), 7.51-7.56 (m, 2H), 7.91 (dt, 1H), 8.23 (d, 1H), 8.69-8.70 (m, 1H), 8.75(s, 1H), 10.51 (s, 1 H).

### Example 9

### Preparation of Compound 1046

Compound #1046 was prepared according to the procedure described for compound #529 using 4-chloromethyl-benzoyl chloride instead of picolinoyl chloride. (3.1 g, 81%). Rf=0.45 in 80:15:5 CH₂Cl₂: EtOAc: MeOH. ¹H NMR CDCl₃δ 0.89 (d, 3H), 0.93 (d, 3H), 1.70 (br s, 2H), 1.79 (br s, 4H), 2.04 (s, 3H), 2.52-2.58 (m, 2H), 2.62-2.63 (m, 1H), 2.76-2.79 (m, 1 H), 3.33 (br s, 7H), 3.43-3.45 (m, 1H), 4.33 (d, 1H), 4.64 (s, 21H), 5.17 (s, 1H), 5.76 (d, 1H), 5.92 (t, 1H), 6.57 (t, 1H), 6.94 (d, 1H), 7.49 (s, 1H), 7.55 (d, 2H), 7.91 (d, 2H), 8.46 (s, 1H), 8.77 (s, 1H).

### Example 10

### Preparation of Compound 1059

To a solution of compound #1046 (0.14 g, 0.2 mmol) in THF (5 ml) were added sodium iodide (30 mg, 0.2 mmol) and morpholine (35 μL, 0.4 mmol). The resulting mixture was heated at reflux for 10 h whereupon it was cooled to room temperature, concentrated under reduce pressure and the residue was redissolved in EtOAc (30 ml), washed with water (10 ml), dried with Na₂SO₄ and concentrated again. The residue was then recrystallized in EtOH (10 ml) to give the compound 1059 as a yellow solid (100 mg, 66%). Rf=0.10 in 80:15:5 CH₂Cl₂:EtOAc:MeOH. ¹H NMR CDCl₃δ 0.93 (s, 3H), 0.95 (d, 3H), 1.70 (br s, 2H), 1.78 (br s, 4H), 2.03 (s, 3H), 2.48 (br s, 4H), 2.55-2.62 (m, 3H), 2.74-2.79 (m, 1H), 3.32 (br s, 7H), 3.45 (m, 1H), 3.59 (s, 2H), 3.72-3.74 (m, 4H), 4.32 (d, 1H), 5.15 (s, 1H), 5.76 (d, 1H), 5.91 (t, 1H), 6.56 (t, 1H), 6.94 (d, 1H), 7.48 (s, 1H), 7.50 (d, 2H), 7.87 (d, 2H), 8.47 (s, 1H), 8.77 (s, 1H).

### Example 11

### Preparation of Compound 1236

Compound #1236 was prepared according to the procedure described for compound #1059 using benzylethyl amine instead of morpholine. Rf=0.43 in 80:15:5 CH₂Cl₂:EtOAc:MeOH. ¹H NMR CDCl₃δ 0.925 (s, 3H), 0.95 (d, 3H), 1.09 (t, 3H), 1.70 (br s, 2H), 1.79 (br s, 4H), 2.04 (s, 3H), 2.50-2.62 (m, 5H), 2.75-2.79 (m, 1H), 3.32 (br s, 7H), 3.46 (m, 1H), 3.59 (s, 2H), 3.63 (s, 2H), 4.33 (d, 1H), 5.16 (s, 1H), 5.78 (d, 1H), 5.91 (t, 1H), 6.57 (t, 1H), 6.94 (d, 1H), 7.25-7.27 (m, 1H), 7.32-7.38 (m, 4H), 7.48 (s, 1H), 7.53 (d, 2H), 7.85 (d, 2H), 8.47 (s, 1H), 8.77 (s, 1H).

### Example 12

### Preparation of Compound 563: 17-(benzoyl)-aminogeldanamycin

A solution of 17-aminogeldanamycin (1 mmol) in EtOAc was treated with Na₂S₂O₄ (0.1 M, 300 mL) at RT. After 2 h, the aqueous layer was extracted twice with EtOAc and the combined organic layers were dried over Na₂SO₄, concentrated under reduce pressure to give 18,21-dihydro-17aminogeldanamycin as a yellow solid. This solid was dissolved in anhydrous THF and transferred via cannula to a mixture of benzoyl chloride (1.1 mmol) and MS4A (1.2 g). Two hours later, EtN(i-Pr)₂ (2.5 mmol) was further added to the reaction mixture. After overnight stirring, the reaction mixture was filtered and concentrated under reduce pressure. Water was then added to the residue which was extracted with EtOAc three times, the combined organic layers were dried over Na₂SO₄ and concentrated under reduce pressure to give the crude product which was purified by flash chromatography to give 17-(benzoyl)-aminogeldanamycin. Rf=0.50 in 80:15:5 CH₂Cl₂:EtOAc:MeOH. Mp=218-220° C. ¹H NMR (CDC₁₃) 0.94 (t, 6H), 1.70 (br s, 2H), 1.79 (br s, 4H), 2.03 (s, 3H), 2.56 (dd, 1H), 2.64 (dd, I H), 2.76-2.79 (m, I H), 3.33 (br s, 7H), 3.44-3.46 (m, 1H), 4.325 (d, I H), 5.16 (s, 1H), 5.77 (d, 1H), 5.91 (t, 1H), 6.57 (t, 1H), 6.94 (d, 1H), 7.48 (s, 1H), 7.52 (t, 2H), 7.62 (t, 1H), 7.91 (d, 2H), 8.47 (s, 1H), 8.77 (s, 1H).

### Example 13

### Preparation of Cell Lysates

Cells for the study were lysed in lysis buffer (20 mM HEPES, pH 7.3, 1 mM EDTA, 5 mM MgCl₂, 100 mM KCl) by manual douncing in a Potter-Elvejem homogenizer.

### Example 14

### HSP90 Lysate Binding Assays

Normal B cell, normal T cell, ZAP70+ CLL B cells and ZAP70− CLL B cells were lysed in lysis buffer as described in Example 13. The lysates were incubated with or without 17-AAG for 30 mins at 4° C., and then incubated with biotin-GM linked to BioMag™ streptavidin magnetic beads (Qiagen) for 1 hr at, 4° C. Tubes were placed on a magnetic rack, and the unbound supernatant removed. The magnetic beads were washed three times in lysis buffer and boiled for 5 min at 95° C. in SDS-PAGE sample buffer. Samples were analyzed on SDS protein gels, and Western blots done using an HSP90 antibody (StressGen, SPA-830). Bands in the Western Blots were quantitated using the Bio-rad Fluor-S MultiImager, and the % inhibition of binding of HSP90 to the biotin-GM was calculated. The IC₅₀ reported is the concentration of 17-AAG needed to cause half-maximal inhibition of binding. The results of competitive binding is showed in FIGS. 1-3.

### Example 15

### Study to Assess the Association of HSP90 with Client Protein

MCF-7 breast carcinoma cells, primary isolates of ZAP70+ and ZAP70− B-cell chronic lymphocytic leukemia (B-CLL) cells and normal T and B cells were lysed as described in Example 13 and co-immunoprecipitation experiments were performed as described in Kamal et al. Nature, 2003 425: 407-410. Protein-A Sepharose beads (Zymed) were pre-blocked with 5%. BSA. The cell lysates were pre-cleared by incubating with 50 μL of protein-A Sepharose beads (50% slurry). To 100 μL of the pre-cleared cell lysate, either no antibody or antibodies to HSP90, p23 and Hop were added, and incubated by rotating for 1 h at 4° C. 50 μL of pre-cleared beads (50% slurry) was then added and incubated by rotating for 1 h at 4° C. Bound beads were briefly centrifuged at 3,000 g and unbound samples collected. Beads were washed thrice in lysis buffer and once with 50 mM Tris, pH 6.8, and then SDS-sample buffer added for 5 min at 95° C. Bound and unbound samples were analysed by SDS-PAGE and western blots using indicated antibodies. The result of the co-immunoprecipitation study is shown in FIG. 4.

### Example 16

### Study to Demonstrate Inhibition of ZAP70 Expression by Selected HSP90 Inhibitors

Primary isolates of ZAP70+ chronic lymphocytic leukemia B-cells from an individual ZAP70+ patient were treated with EC1 (17-AAG), EC116 (an inactive structurally-related HSP90 inhibitor) or EC82 or EC86 (two other known HSP90 inhibitors) for 24 hours at 37° C. Levels of ZAP70 protein expression were measured by indirect immunofluorescence of permeabilized cells with specific anti-ZAP70 antibodies and FACS analysis. Result of the study is shown in FIG. 5. All three active HSP90 inhibitors dose-dependently induced degradation of ZAP70, confirming that ZAP70 is an HSP90-dependent client protein, as was indicated by the physical association demonstrated in the co-immunoprecipitation experiments (FIG. 4). The fact that three structurally-unrelated HSP90 inhibitors produced the same effect strongly implicates HSP90 as an essential protein for the stability of ZAP70 in CLL B-cells.

### Example 17

### Study to Determine Downstream Effect of Inhibiting HSP90 on Blood Cells of ZAP70+ CCL B-Cell Patient

Primary isolates of white blood cells from an individual ZAP70+ chronic lymphocytic leukemia B-cell patient were left untreated or treated with 300 nM 17-AAG for 24 hours at 37° C. The samples were than prepared for flow cytometry analysis by a method described in Rassebti et al. supra. The cells were first stained with CD19-specific and CD3-specific monoclonal antibodies conjugated with allophycocyanin and phycoerythrin, respectively (Pharmingen), and later stained with a monoclonal antibody specific for ZAP70 that has been conjugated to Alexa-488 dye (Becton Dickinson). CD3 is a specific marker of T cells. Levels of ZAP70 protein expression were measured by flow cytometry (FACSCalibur, BD Biosciences) and Flow-Jo software, version 2.7.4 (Tree Star). The result is documented in FIG. 6, the left panel shows the ZAP70 expression in untreated cells, and the right panel shows the ZAP70 expression of the untreated cells.

In the untreated cells (left panel), approximately 5% of the cells were normal T-cells (CD3+, ZAP70+, upper right quadrant) and ˜85% of the cells were ZAP70+ CLL B-cells (CD3−, ZAP70+, lower right quadrant). 17-AAG induced degradation of ZAP70 in the CLL B-cells (% positive cells 85%→34%), but not in the normal T-cells (% positive cells 4.5%→4.2%), as predicted from the physical association demonstrated in CLL B-cells, but not in the normal T-cells in the co-immunoprecipitation experiments (FIG. 4).

### Example 18

### The Concentration Dependent Effect of Inhibiting HSP90 on ZAP70+ CCL B Cell Viability

Primary isolates of ZAP70+ chronic lymphocytic leukemia B-cells from an individual patient were treated with increasing concentration of EC1 (17-AAG) or EC116 (inactive structurally-related HSP90 inhibitor) for 48 hours at 37° C. Apoptotic cells were identified by a standard protocol using the mitochondrial vital dye DiOC6 and propidium iodide staining. Results were plotted in FIG. 7 of the % viability vs. concentration of the inhibitor in nM. The % viability is expressed as 100%-% apoptotic cells. ZAP70+ tumor cells were readily killed by 17-AAG, with a 50% inhibitory concentration (IC₅₀) of approximately 80 nM.

### Example 19

### The Time Dependent Effect of Inhibiting HSP90 on ZAP70+ CCL B Cell Viability

Primary isolates of B-cell chronic lymphocytic leukemia cells from an individual ZAP70+ patient were treated with 100 nM EC1 (17-AAG) or EC116 (inactive structurally-related HSP90 inhibitor) for varying times at 37° C. Apoptotic cells were identified by a standard protocol using the mitochondrial vital dye DiOC6 and propidium iodide staining. Results of the study were plotted in FIG. 8 of the % viability vs. treatment time in hours. The % viability is expressed as 100%-% apoptotic cells. ZAP70+ tumor cells were rapidly killed by 17-AAG, with a 50% of the cells succumbing in approximately 48 hours.

### Example 20

### Downstream Effect of Inhibiting HSP90 in CLL B Cells

Primary isolates of chronic lymphocytic leukemia B-cells (CLL B cells) from sixteen ZAP70+ patients and eleven ZAP70− patients were treated with 100 nM of 17-AAG for 48 hours at 37° C. Apoptotic cells were identified by a standard protocol using the mitochondrial vital dye DiOC6 and propidium iodide staining. Results of the study were plotted in FIG. 9. The % viability is expressed as 100%-% apoptotic cells. ZAP70+ tumor cells were readily killed by 17-AAG, with an average % survival of 45.74+/−3.177%, whereas ZAP70− cells were unaffected by the drug under the same conditions—survival in these cells was 93+/−1.701%. The Students T-Test of the difference in survival has a P-value of <0.0001, which is highly statistically significant.

The foregoing examples are not intended to be limiting of and are merely representative of various embodiments of the invention. It will be readily apparent to one skilled in the art that varying substitutions and modifications may be made to the invention without departing from the scope and spirit of the invention. Thus, such additional embodiments are within the scope of the invention and the following claims.

Antibodies, polyclonal or monoclonal, can be purchased from a variety of commercial suppliers, or may be manufactured using well-known methods, e.g., as described in Harlow et al., ANTIBODIES: A LABORATORY MANUAL, 2nd Ed., Cold Spring Harbor Laboratory Press, Cold Spring Harbor, N.Y. (1988). The reagents described herein are either commercially available, e.g., from Sigma-Aldrich, or else readily producible without undue experimentation using routine procedures known to those of ordinary skill in the art and/or described in publications herein incorporated by reference.

What is claimed is:

1. A method of treating a form of chronic lymphocytic leukemia characterized by elevated levels of ZAP70 expression in B cells, comprising administering to a patient in need of such treatment a pharmaceutically effective amount of a Hsp90 inhibitor.

2. The method of claim 1, wherein said inhibitor is an ansamycin.

3. The method of claim 2, wherein said ansamycin is selected from the group below, or a polymorph, solvate, ester, tautomer, enantiomer, pharmaceutically acceptable salt or prodrug thereof:

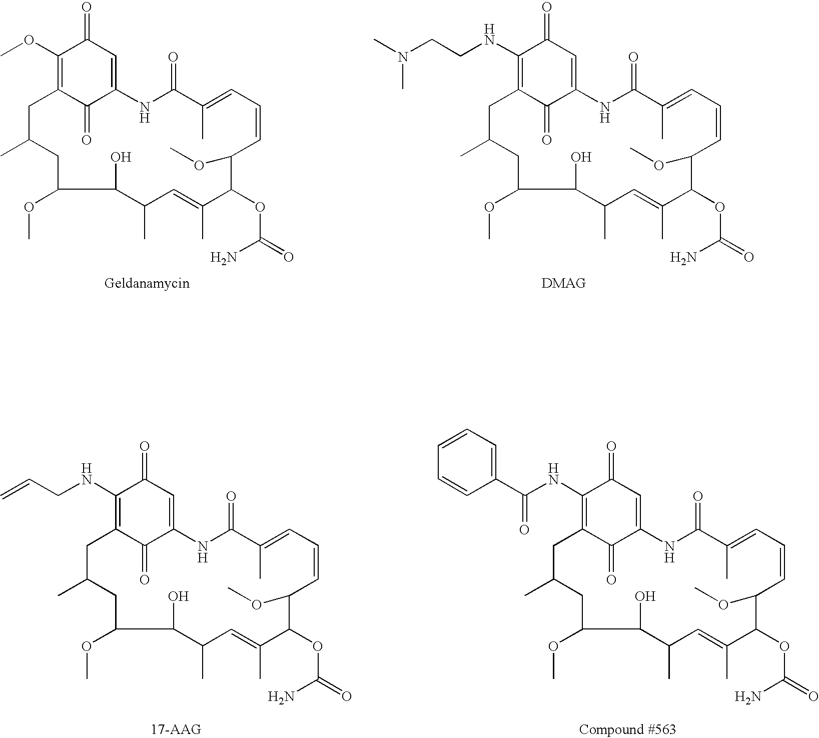

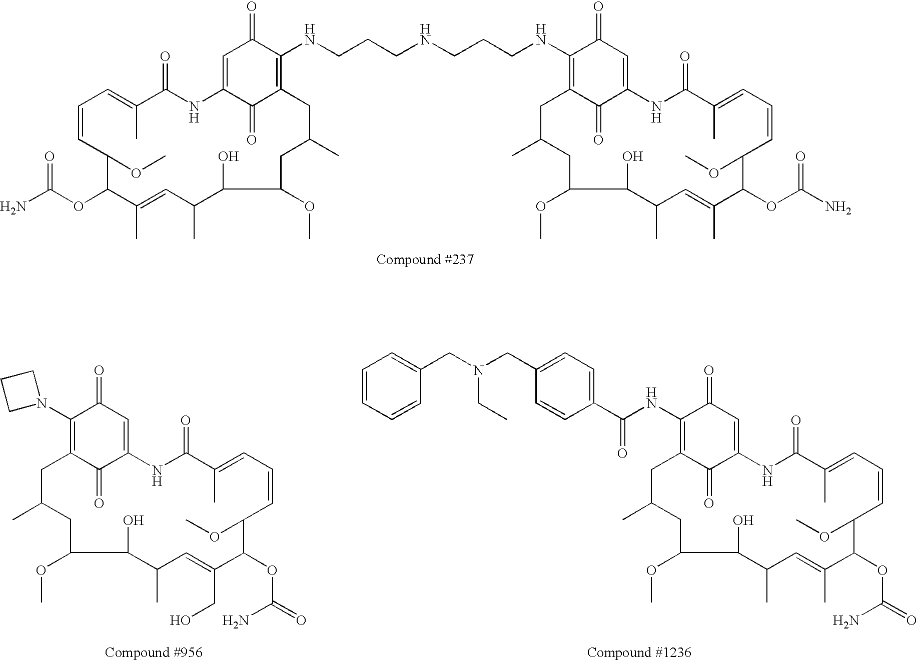

4. The method of claim 2 wherein said ansamycin is 17-AAG.

5. The method of claim 2 wherein said ansamycin comprises low melt forms of 17-AAG characterized by DSC melting temperatures below 175° C.

6. The method of claim 4 wherein said 17-AAG is selected from a high melt form, a low melt form, an amorphous form, or combination thereof.

7. The method of claim 1, wherein said inhibitor binds at the ATP-binding site of a HSP90.

8. The method of claim 1 wherein said administering is intralesional.

9. The method of claim 1 wherein said administering is parenteral.

10. The method of claim 1 wherein said administering is oral.

11. The method of claim 1 wherein said administering is intraveneous.

12. The method of claim 1 wherein said HSP90 inhibitor has an IC₅₀ at least two-fold lower for said HSP90 in the B cells of said patient having elevated ZAP70 than for B cells that do not have elevated ZAP70.

13. The method of claim 1 wherein said HspP90 inhibitor has an IC₅₀ at least five-fold lower for said HSP90 in the B cells of said patient having elevated ZAP70 than for B cells that do not have elevated ZAP70.

14. The method of claim 1 wherein said HSP90 inhibitor has an IC₅₀ at least ten-fold lower for said HSP90 in the B cells of said patient having elevated ZAP70 than for B cells that do not have elevated ZAP70.

15. The method of claim 1 wherein said inhibitor exhibits an IC₅₀ of about 100 nM or less for the HSP90 in the B cells having elevated ZAP70.

16. The method of claim 1 wherein said inhibitor exhibits an IC₅₀ of about 75 nM or less for the HSP90 in the B cells having elevated ZAP70.

17. The method of claim 1 wherein said inhibitor exhibits an IC₅₀ of about 50 nM or less for the HSP90 in the B cells having elevated ZAP70.

18. The method of claim 1 wherein said inhibitor exhibits an IC₅₀ of about 30 nM for the HSP90 in the B cells having elevated ZAP70.
